(19) United States
(12) Patent Application Publication
(10) Pub. No.: US 2019/0091114 A1
(43) Pub. Date: Mar. 28, 2019

(54) FACIAL MASK SET

(71) Applicant: OUTINFUTURES CO., LTD., Seoul (KR)

(72) Inventor: Youngwook CHOI, Seoul (KR)

(21) Appl. No.: 16/198,958

(22) Filed: Nov. 23, 2018

Related U.S. Application Data
(63) Continuation of application No. PCT/KR2018/008220, filed on Jul. 20, 2018.

(30) Foreign Application Priority Data
Jul. 21, 2017 (KR) 10-2017-0092779
Aug. 14, 2017 (KR) 10-2017-0103030
Sep. 29, 2017 (KR) 10-2017-0127262

(51) Int. Cl. A61K 8/02; A61K 8/44; A61K 8/60; A61K 8/49; A61K 8/34; A61K 8/92; A61Q 19/02; A61Q 19/08

(52) U.S. Cl. CPC A61K 8/0212; A61K 8/44; A61K 8/606; A61K 8/4926; A61K 8/345; A61K 2800/87; A61Q 19/02; A61Q 19/08; A61K 2800/524; A61K 2800/48; A61K 2800/88; A61K 8/922

(57) ABSTRACT

The present invention relates to a peel-off facial mask cosmetic composition, comprising: a liquid-phase first reagent containing purified water and a liquid-phase second reagent containing an oily emollient, a surfactant, a gel forming agent, a gelling agent and a gel retarder, wherein the gel forming agent, the gelling agent and the gel retarder contained in the second reagent are not gelled by the oily emollient and are present in an inactive state.

### CROSS-REFERENCE TO RELATED APPLICATIONS

This application is a continuation of International Patent Application No. PCT/KR2018/008220 with a filing date of Jul. 20, 2018, designating the United States, now pending, and further claims priority to Korean Patent Application No. 10-2017-0092779 with a filing date of Jul. 21, 2017, Korean Patent Application No. 10-2017-0103030 with a filing date Aug. 14, 2017, Korean Patent Application No. 10-2017-0127262 with a filing date Sep. 29, 2017. The content of the aforementioned applications, including any intervening amendments thereto, are incorporated herein by reference.

### TECHNICAL FIELD

The present invention relates to a facial mask set.

### BACKGROUND ART

With the development of modern society, not only women but also men's attention to skin beauty is concentrated. In particular, there is increasing interest in preventing skin aging by maintaining skin elastic and clean, and various products for improving the skin are available currently on the market.

In this respect, the human skin, comprising epidermis, dermis and subcutaneous tissue, is an important part of the role of protecting the human body from environmental factors such as external stimuli or pollution.

The outermost layer of the epidermis is stratum corneum, which is the portion in direct contact with cosmetics, and also is a portion capable of displaying the state of the skin. Through the keratinization process of epidermal cells, the outermost keratinocytes repeatedly shed and form, and within a day, millions of dead keratinocytes are shed from the skin and replaced by new keratinocytes.

If the keratinization process is not smooth and the keratinocyte formation is abnormal or the keratinocytes do not well shed, the skin looks dull and does not easily make up or looks inactive.

Therefore, people provide sufficient nutrition and moisturization to the epidermis of the skin, and use various facial mask products to make the stratum corneum shed smoothly.

Generally, facial mask products exist in various dosage forms such as a cream type, a gel type, a powder type, and a mask type, and are classified into a peel-off type, a rinse-off type, and a rinse-free type according to the method of use.

In facial mask products of the types described above, after the liquid of a peal-off product or a gel product is applied to the skin, the nutrient of the facial mask is absorbed into the skin over time.

The product applied to the skin surface itself is dried to form a film, and the film has the advantage that it is easy to peel off without washing with water.

In particular, with the peeling of the dry film, the peel-off product can remove sebum or various wastes out of the stratum corneum and pores. In other words, the peel-off product not only supplies nutrients, but also has the effect of removing immediate pollutants.

Conventional facial mask products described above usually have a first reagent of a powder dosage form and a liquid-phase second reagent to be mixed in a separate container for use.

In this case, there is a problem that since the first reagent is of a powder dosage form, it takes more time and effort to mix the first reagent with other liquid-phase second reagents.

Further, there is a problem that even if the user mixes the first reagent and the second reagent with a predetermined time and effort, the first reagent of the powder dosage form and the liquid-phase second reagent cannot be sufficiently mixed, thereby failing to exert the originally expected effect.

### SUMMARY OF THE INVENTION

### Technical Problem

An object of the present invention is to provide a facial mask set to solve the problem of unnecessary time and effort spent by the user in mixing and using the first reagent and the second reagent.

Another object of the present invention is to provide a facial mask set to solve the problem that even if the user mixes the first reagent and the second reagent with a predetermined time and effort, since the first reagent of the powder dosage form and the liquid-phase second reagent cannot be mixed sufficiently, the originally expected effect can not be achieved.

Yet another object of the present invention is to provide a facial mask set to solve the inconvenience of mixing a first reagent of a powder dosage form and a liquid-phase second reagent in a separate container.

### Means for Solving the Problems

To solve the problems mentioned above, the present invention can provide a peel-off facial mask cosmetic composition, comprising: a liquid-phase first reagent containing purified water and a liquid-phase second reagent containing an oily emollient, a surfactant, a gel forming agent, a gelling agent and a gel retarder,

wherein the gel forming agent, the gelling agent and the gel retarder contained in the second reagent are not gelled by the oily emollient and are present in an inactive state.

Further, a peel-off facial mask cosmetic composition can be provided, wherein the ratio of the first reagent to the second reagent is 95:5 to 50:50 by volume.

Further, a peel-off facial mask cosmetic composition can be provided, wherein the content of the purified water is 70 wt % or more based on the total weight of the first reagent, the content of the oily emollient is 65-95 wt % based on the total weight of the second reagent, and the content of the surfactant is 1-25 wt % based on the total weight of the second reagent.

Further, it is possible to provide a peel-off facial mask cosmetic composition, wherein the oily emollient is an oil.

Further, a peel-off facial mask cosmetic composition can be provided, wherein the first reagent further comprises at least one selected from a group consisting of a metal ion chelating agent, a wrinkle removal functional raw material, a whitening functional raw material, a humectant, a surfactant, and a preservative.

Further, a peel-off facial mask cosmetic composition can be provided, wherein based on the total weight of the first reagent, the content of the metal ion chelating agent is 0.001-1 wt %, the content of the wrinkle removal functional raw material is 0.02-0.06 wt %, the content of the whitening functional raw material is 1.8-2.2 wt %, the content of the humectant is 0.1-25 wt %, and the content of the surfactant is 0.001-5 wt %.

Further, a peel-off facial mask cosmetic composition, wherein the metal ion chelating agent is one selected from a group consisting of condensed phosphate, ethylenediaminetetraacetic acid (EDTA), disodium EDTA, nitrilotriacetic acid, dihydroxyethylglycine, gluconic acid, citric acid, and tartaric acid;

the wrinkle removal functional material is one selected from a group consisting of adenosine, retinol, retinyl palmitate and polyethoxylated retinamide;

the whitening functional raw material is one selected from a group consisting of niacinamide, broussonetia extract, ascorbyl glucoside, ascorbyl tetraisopalmitate, arbutin, α-Bisabolol, ethyl ascorbyl ether, and oil soluble licorice (glycyrrhiza) extract;

the humectant is one selected from a group consisting of glycerin, propylene glycol, dipropylene glycol, butanediol, isopropyl glycol, pentanediol, arginine, and mixtures thereof;

the surfactant is one selected from a group consisting of polyglycerol fatty acid esters, polyethylene glycol fatty acid esters, polyoxyethylene hydrogenated castor oil, polyoxyethylene alkyl ethers, polyoxyethylene glycerin fatty acid esters, polyoxyethylene polyoxypropylene alkyl ethers, and mixtures thereof.

Further, a peel-off facial mask cosmetic composition can be provided, wherein the metal ion chelating agent is disodium EDTA; the wrinkle removal functional raw material is adenosine; the whitening functional raw material is niacinamide; the humectant is dipropylene glycol; the surfactant is PEG-60 hydrogenated castor oil.

Further, a peel-off facial mask cosmetic composition can be provided, wherein the second reagent further comprises at least one component selected from a group consisting of a preservative and a thickener.

Further, a peel-off facial mask cosmetic composition can be provided, wherein based on the total weight of the second reagent, the content of the thickener is 0.5-20 wt %, the content of the gel forming agent is 1-25 wt %, the content of the gel retarder is 0.01-10 wt %, and the content of the gelling agent is 0.01-10 wt %.

Further, a peel-off facial mask cosmetic composition can be provided, wherein the surfactant is one selected from a group consisting of polyglycerol fatty acid ester fatty acid esters, polyoxyethylene hydrogenated castor oil, polyoxyethylene alkyl ethers, polyoxyethylene glycerin fatty acid esters, polyoxyethylene polyoxypropylene alkyl ethers, and mixtures thereof;

the thickener is at least one selected from a group consisting of waxes, polymers, and silicas.

Further, a peel-off facial mask cosmetic composition can be provided, wherein the surfactant is PEG-8 glyceryl isostearate; and the thickener is silica dimethylsilane ester.

Moreover, the present invention can further provide a facial mask set, comprising: a first body providing a space for accommodating a first reagent; a second body extending over or below the first body and providing a space for accommodating a second reagent; and a discharge portion for discharging a third reagent formed by mixing the first reagent and the second reagent;

the first reagent is provided in a liquid phase containing purified water;

the second reagent is provided in a liquid phase containing an oily emollient, a gel forming agent, a gelling agent, and a gel retarder;

wherein the gel forming agent, the gelling agent and the gel retarder contained in the second reagent are not gelled by the oily emollient and are present in an inactive state.

Further, a facial mask set can be provided, wherein the first body is disposed on an outer peripheral surface of the first body and comprises a first seal portion for sealing the first reagent;

the second body is disposed on an outer peripheral surface of the second body and comprises a second seal portion for sealing the second reagent.

Further, a facial mask set can be provided, wherein the first seal portion comprises a seal portion 1-1 that communicates with the second seal portion and a seal portion 1-2 that does not communicate with the second seal portion;

the second seal portion comprises a seal portion 2-1 that communicates with the first seal portion and a seal portion 2-2 that does not communicate with the first seal portion.

Further, a facial mask set can be provided, wherein the sealing strength of the seal portion 1-1 and the seal portion 2-1 is smaller than the sealing strength of the seal portion 1-2 and the seal portion 2-2.

Further, a facial mask set can be provided, wherein the discharge portion comprises: a discharge flow path for providing a flow path in which the third reagent flows, and a discharge opening portion which opens one end of the discharge flow path;

the cross-sectional area of an inflow portion of the discharge flow path into which the third reagent flows is larger than the cross-sectional area of an outflow portion from which the third reagent flows out.

Further, a facial mask set can be provided, wherein the discharge portion is formed by cutting a first cut portion provided on a right side or a left side surface of the facial mask set and a second cut portion provided on an upper surface or a lower surface of the facial mask set.

Further, a facial mask set can be provided, wherein a lower surface of the second body is formed into a downwardly protruding shape;

the discharge portion is formed by cutting a first cut portion provided on a left side of the facial mask set and a second cut portion provided on a right side of the facial mask set, and the discharge portion penetrates through the lower part of the second body.

Moreover, the present invention can also provide a facial mask set, comprising: a first body for accommodating a first reagent; a second body for accommodating a second reagent; and a third body for providing a space for a third reagent formed by mixing the first reagent and the second reagent;

wherein after the first reagent and the second reagent are injected into the third body, the third reagent is formed by a relative displacement change of the first reagent and the second reagent, and the third reagent is a cream-type emulsion with viscosity higher than the viscosity of the first reagent and the second reagent and is gelled over time;

the first reagent is provided in a liquid phase containing purified water;

the second reagent is provided in a liquid phase containing an oily emollient, a gel forming agent, a gelling agent, and a gel retarder;

wherein the gel forming agent, the gelling agent and the gel retarder contained in the second reagent are not gelled by the oily emollient and are present in an inactive state.

Moreover, the present invention can further provide a facial mask set, comprising: a first body for accommodating a first reagent; and a second body for accommodating the second reagent and providing a space for a third reagent formed by mixing the first reagent and the second reagent;

wherein after the first reagent is injected into the second body, the third reagent is formed by a relative displacement change of the first reagent and the second reagent, and the third reagent is a cream-type emulsion with viscosity higher than the viscosity of the first reagent and the second reagent and is gelled over time;

the first reagent is provided in a liquid phase containing purified water;

the second reagent is provided in a liquid phase containing an oily emollient, a gel forming agent, a gelling agent, and a gel retarder;

wherein the gel forming agent, the gelling agent and the gel retarder contained in the second reagent are not gelled by the oily emollient and are present in an inactive state.

Further, a facial mask set can be provided, wherein the relative displacement change of the first reagent and the second reagent is generated by agitation of the third body.

Further, a facial mask set can be provided, wherein the relative displacement change of the first reagent and the second reagent is generated by the movement action of an external displacement change generating means.

Further, a facial mask set can be provided, wherein the third body comprises: a third body accommodating portion that accommodates the first reagent and the second reagent; and a third body opening and closing portion that opens or closes on one side of the accommodating portion.

### Effects of the Invention

Before use of the peel-off facial mask cosmetic composition of the present invention, a first reagent containing purified water or a functional cosmetic liquid and a second reagent containing an oily emollient and a surfactant are simply mixed and emulsified and gelled within a short time. The gel has excellent strength and can be applied to the skin to effectively remove waste and dead skin from the skin with reduced physical irritation to the skin, and has good effects of lifting, whitening and moisturizing the skin.

The curing time of the gel can be adjusted by mixing the first reagent and the second reagent before use. Since the liquid-phase first reagent and the liquid-phase second reagent are mixed, it is possible to prevent the problem of precipitation or uniform mixing when a powder and a liquid phase are mixed, and the effects of easy mixing, time saving, and quick skin care are achieved.

In addition, a facial mask set can be provided, wherein both the first reagent and the second reagent are provided in a form of liquid phase, thereby preventing the user from taking unnecessary time and effort in mixing and using the first reagent and the second reagent.

Further, the present invention can provide a facial mask set, wherein both the first reagent and the second reagent are provided in a form of liquid phase such that the first reagent and the second reagent are sufficiently mixed, and the desired effect can be sufficiently exerted.

In addition, the present invention can provide a facial mask set, since the liquid-phase first reagent and the liquid-phase second reagent can be mixed in a state of being accommodated in one container, so that the inconvenience brought when the user can mix a first reagent and a liquid-phase second reagent in a separate container can be solved.

### BRIEF DESCRIPTION OF DRAWINGS

FIG. 1 shows a front view of a facial mask set according to Embodiment 1.

FIG. 2 shows a front view of a facial mask set according to Embodiment 2.

FIG. 3 shows a front view of a facial mask set according to Embodiment 3.

FIG. 4 shows a side view of the facial mask set according to Embodiment 3.

FIG. 5 shows a front view of a facial mask set according to Embodiment 4.

FIG. 6 shows a side view of the facial mask set according to Embodiment 4.

FIG. 7 shows an example of a discharge flow path of the facial mask set according to Embodiment 3 and Embodiment 4.

FIG. 8 shows another example of the discharge flow path of the facial mask set according to Embodiment 3 and Embodiment 4.

FIG. 9 shows a first body, a second body, and a third body of a facial mask set according to Embodiment 5.

FIG. 10 shows the facial mask set according to Embodiment 5 in which the first reagent and the second reagent accommodated in the third body are mixed.

FIG. 11 (a) shows a conventional peel-off mask cosmetic composition in which a powder and a liquid phase are mixed.

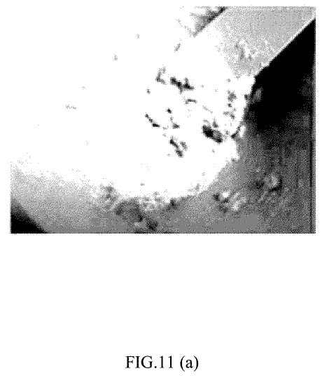

FIG. 11 (b) shows a peel-off mask cosmetic composition obtained by mixing the liquid-phase first reagent and the liquid-phase second reagent of the present invention.

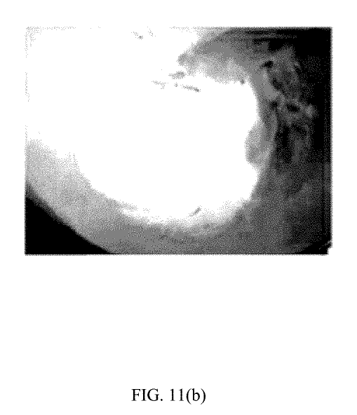

FIG. 12 is a photograph showing the composition of Embodiment 1, wherein FIG. 12 (a) shows the liquid-phase first reagent and the liquid-phase second reagent before mixing, and FIG. 12 (b) shows that the liquid-phase first reagent and the liquid-phase second reagent are mixed and gelled.

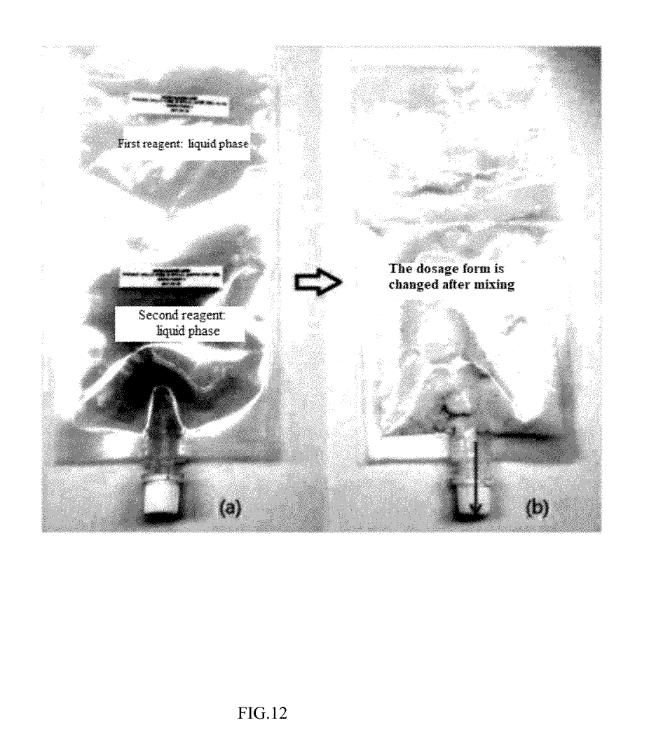

### DETAILED DESCRIPTION OF SPECIFIC EMBODIMENTS

Hereinafter, preferred embodiments according to the present invention will be described in detail with reference to the accompanying drawings. The detailed description below, as well as the accompanying drawings, is intended to illustrate the exemplary embodiments of the invention, but not intended to represent the only embodiments in which the present invention may be practiced.

These embodiments are described solely to fully disclose the present invention, and are provided with an intention to completely inform those skilled in the art to which the invention pertains of the scope of the invention.

In some cases, to avoid obscuring the concept of the present invention, well-known structures and devices may be omitted, and may be shown in block diagram form centering on the core functions of the structures and devices. Also, the same components are denoted by the same reference numerals throughout the description.

Throughout the description, when a component of a certain portion is referred to as “comprising or including”, it means that the portion may also comprise/include other components, and other components are not excluded unless otherwise specifically stated.

Additionally, the idiomatic expression “ . . . portion” described in the description may indicate a unit for processing at least one function or operation. In addition, the terms “a/an” and “one” and other similar relevant words should be interpreted as covering both singular and plural cases, unless otherwise indicated in the context of the description of the present invention or constituting an obvious grammatical contradiction.

Further, specific terms used in the embodiments of the present invention are provided for the convenience of understanding the present invention. Unless otherwise defined, all terms, including technical and scientific terms, used herein have the same meanings as commonly understood by one of ordinary skill in the art to which the present invention pertains. The use of such specific terms may be changed to other forms without departing from the spirit of the present invention.

In order to facilitate the description of the facial mask set of embodiments, it will be described in a total of four embodiments.

More specifically, according to the type of the discharge portion that accommodates the first reagent and the second reagent inside the facial mask set, the four embodiments are classified into Embodiment 1 and Embodiment 2 of the type having a discharge flow path generated by cutting the lower end portion of the facial mask set and Embodiment 3 and Embodiment 4 of the type having a discharge opening portion communicating with the lower end portion of the facial mask set.

However, this is for explaining an embodiment. The facial mask set of the present invention is only provided with a discharge portion, so that the first reagent and the second reagent accommodated inside the facial mask set are discharged to the outside of the facial mask set, and the user can change the shape of the discharge portion to the same type as Embodiments 1-4 or varied and implemented into other various types, which does not limit the right scope of the facial mask set of the present invention.

Next, the facial mask sets of Embodiments 1-4 will be sequentially described.

### Embodiment 1

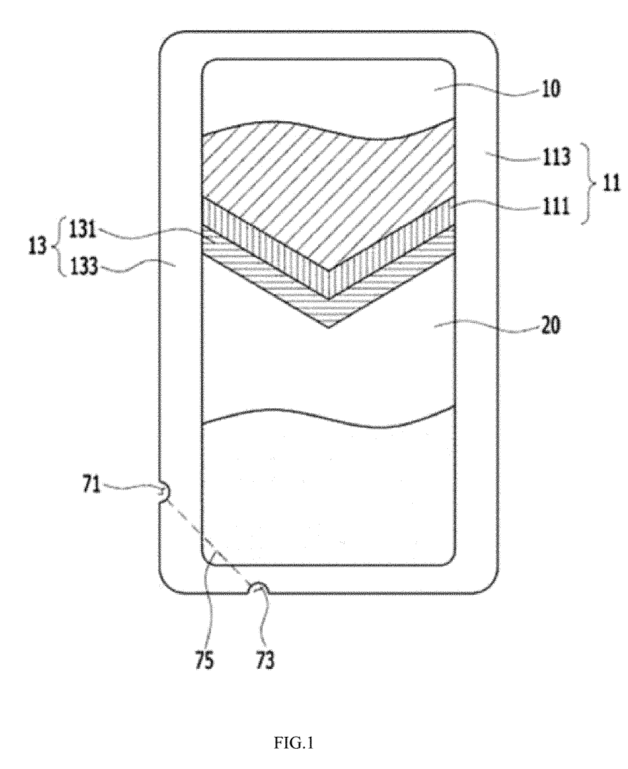

FIG. 1 shows a front view of a facial mask set according to Embodiment 1.

Referring to FIG. 1, the facial mask set of Embodiment 1 comprises: a first body (10) that seals and accommodates a first reagent (40) and a second body (20) extending over or below the first body and configured to seal and accommodate a second reagent (50).

The first body (10) is disposed on an outer peripheral surface of the first body (10) and comprises a first seal portion (11) for sealing the first reagent (40), and the second body (20) is disposed on an outer peripheral surface of the second body (20) and comprises a second seal portion (13) for sealing the second reagent (50).

The first seal portion (11) comprises a seal portion 1-1 (111) communicating with the second seal portion (13) and a seal portion 1-2 (113) not communicating with the second seal portion (13). The second seal portion (13) comprises a seal portion 2-1 (131) communicating with the first seal portion (11) and a seal portion 2-2 (133) not communicating with the first seal portion (11).

In this case, the sealing strength of the seal portion 1-1 (111) and the seal portion 2-1 (131) is smaller than the sealing strength of the seal portion 1-2 (113) and the seal portion 2-2 (133).

This is because the seal portion 1-2 (113) and the seal portion 2-2 (133) are not broken when the user presses the facial mask set from the outside, and the seal portion 1-1 (111) and the second seal portion 2-1 (131) are disposed to communicate with each other by a predetermined pressure applied externally.

More specifically, the first reagent (40) disposed in the first body (10) and the second reagent (50) disposed in the second body (20) are separated from each other before use by the user. When using the facial mask set, the user applies a pressure to the first body (10) or the second body (20) with hands, and the seal portion 1-1 (111) and the seal portion 2-1 (131) having a seal strength smaller than the above pressure are broken, thus forming a flow path in which the first reagent (40) in the first body (10) moves toward the second body (20) or the second reagent (50) in the second body (20) moves toward the first body (10); the first body (10) is in communication with the second body (20), and the first reagent (40) and the second reagent (50) are mixed in the first body (10) or the second body (20).

Therefore, before use of the facial mask set in this embodiment, the first reagent (40) and the second reagent (50) can be separately stored in the first body (10) and the second body (20); when use of the facial mask set, a separate tool is not required, thus providing the convenience in mixing the first reagent (40) and the second reagent (50) in the facial mask set.

Further, the facial mask set in this embodiment further comprises a cut portion for forming a discharge flow path for discharging a third reagent (60) formed by mixing the first reagent (40) and the second reagent (50) to the outside.

The cut portion comprises a first cut portion (71) formed on the right side or the left side of the facial mask set, and a second cut portion (73) formed on the upper surface or the lower surface of the facial mask set.

More specifically, the first cut portion (71) may be disposed below the center of the left side of the facial mask set, and correspondingly, the second cut portion (73) may be disposed on the left side with respect to the center of the lower end portion. A virtual perforation line (75) formed by connecting the first cut portion (71) and the second cut portion (73) may form a diagonal line.

However, this is an example for convenience of explanation, and the first cut portion (71) may be disposed not only on the left side of the facial mask set but also on the right side, and the second cut portion (73) may be disposed on the lower surface of the facial mask set but also on the upper surface, and the first cut portion (71) and the second cut portion (73) are shaped to satisfy the condition that the virtual perforation line (75) connecting the first cut portion (71) and the second cut portion (73) obliquely cuts the edge of the second body (20) so that the third reagent (60) is discharged in a small amount, and the user can make various changes and implementations in the locations of the first cut portion (71) and the second cut portion (73) as needed, which does not limit the scope of the present invention.

### Embodiment 2

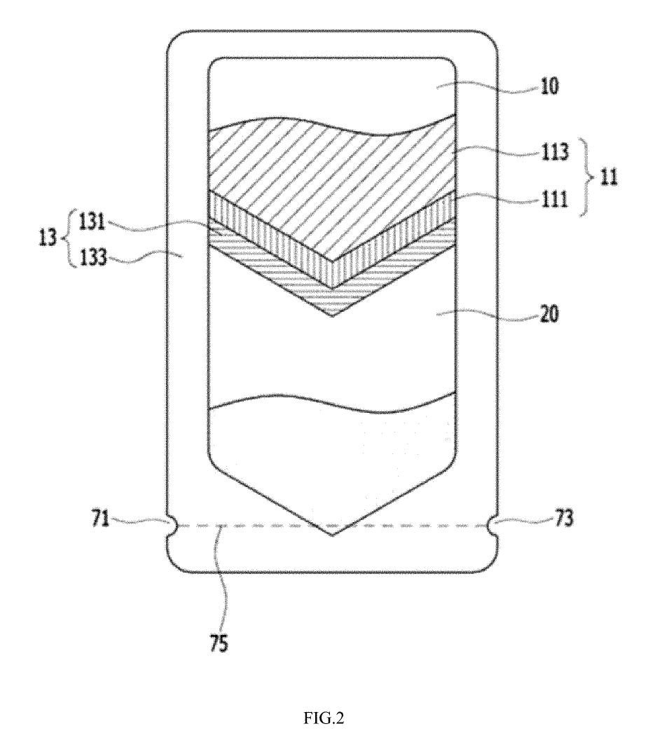

FIG. 2 shows a front view of a facial mask set according to Embodiment 2.

The facial mask set in Embodiment 2 differs from the facial mask set in Embodiment 1 only in the shape of the lower end portion of the second body (20) and the shape of the cut portions (71, 73), and has the rest of the configuration substantially the same as Embodiment 1, and therefore the repetitive description will be omitted, and only the differences will be explained.

Since the lower end portion of the second body (20) of the facial mask set of Embodiment 1 is flat, the virtual perforation line (75) connecting the cut portions (71, 73) is obliquely formed, thereby controlling the discharge amount of the third reagent (60) discharged from the second body (20), and conversely, the lower end portion of the second body (20) of the facial mask set of Embodiment 2 may be arranged into a downwardly protruding “V” shape.

In order to control the discharge amount of the third reagent (60) in the second body (20), the lower end of the second body (20) is preferably cut flat.

Therefore, the first cut portion (71) of the facial mask set of Embodiment 2 is disposed below the center of the left side of the facial mask set, and the second cut portion (73) is disposed below the center of the right side of the facial mask set. The virtual perforation line (75) connecting the first cut portion (71) and the second cut portion (73) is parallel to the lower end portion of the facial mask set, and is provided to cut a part of the protruding portion of the second body (20).

Therefore, an effect that the user can easily discharge the third reagent (60) from the facial mask set is achieved.

However, this is an example shown for convenience of explanation only, and the lower end portion of the second body (20) may be shaped to downwardly protrude, and the shape of the lower end portion is not limited to the “V” shape described above, and the user may make various variants and implementations of the shape of the lower end portion of the second body (20) as needed, which does not limit the scope of the present invention.

### Embodiment 3

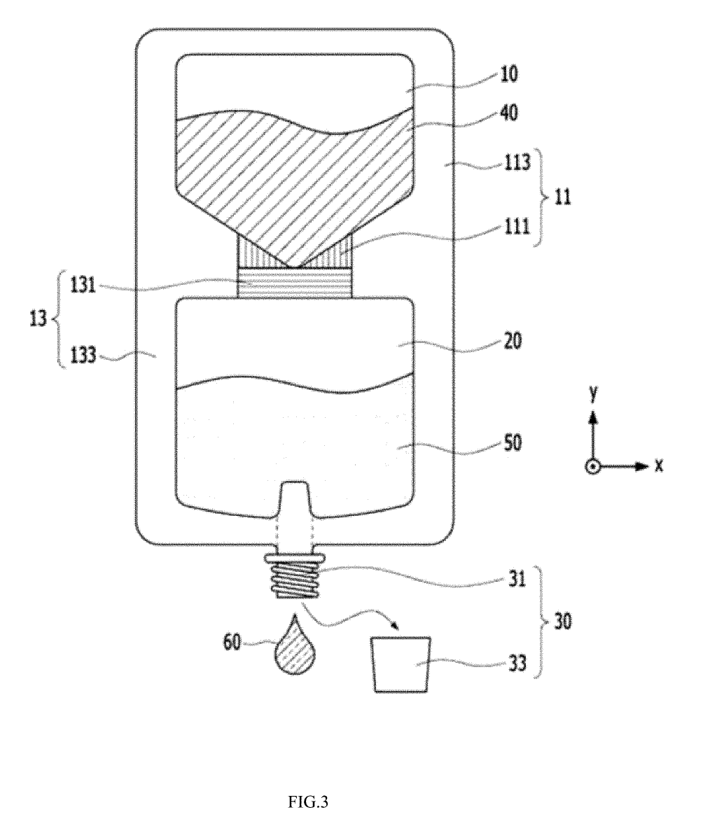

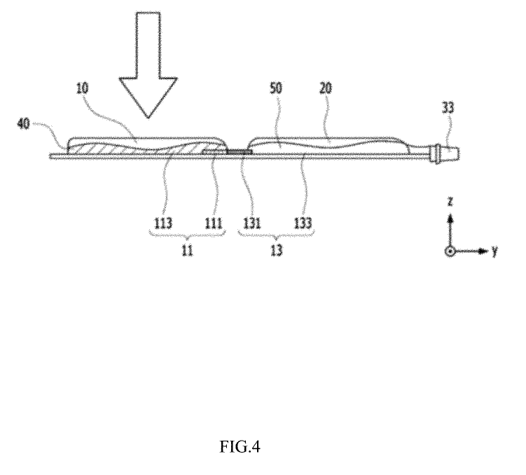

FIG. 3 shows a front view of a facial mask set according to Embodiment 3, and FIG. 4 shows a side view of the facial mask set according to Embodiment 3.

Referring to FIG. 3 and FIG. 4, the facial mask set of Embodiment 3 comprises: a first body (10) that seals and accommodates a first reagent (40) and a second body (20) that seals and accommodates a second reagent (50).

The first body (10) is disposed on an outer peripheral surface of the first body (10) and comprises a first seal portion (11) for sealing the first reagent (40), and the second body (20) is disposed on an outer peripheral surface of the second body (20) and comprises a second seal portion (13) for sealing the second reagent (50).

The first seal portion (11) comprises a seal portion 1-1 (111) communicating with the second seal portion (13) and a seal portion 1-2 (113) not communicating with the second seal portion (13). The second seal portion (13) comprises a seal portion 2-1 (131) communicating with the first seal portion (11) and a seal portion 2-2 (133) not communicating with the first seal portion (11).

In this case, the sealing strength of the seal portion 1-1 (111) and the seal portion 2-1 (131) is smaller than the sealing strength of the seal portion 1-2 (113) and the seal portion 2-2 (133).

This is because the seal portion 1-2 (113) and the seal portion 2-2 (133) are not broken when the user presses the facial mask set from the outside, and the seal portion 1-1 (111) and the second seal portion 2-1 (131) are disposed to communicate with each other by a predetermined pressure applied externally.

More specifically, the first reagent (40) disposed in the first body (10) and the second reagent (50) disposed in the second body (20) are separated from each other before use by the user. When using the facial mask set, the user applies a pressure to the first body (10) or the second body (20) with hands, and the seal portion 1-1 (111) and the seal portion 2-1 (131) having a seal strength smaller than the above pressure are broken, thus forming a flow path in which the first reagent (40) in the first body (10) moves toward the second body (20) or the second reagent (50) in the second body (20) moves toward the first body (10); the first body (10) is in communication with the second body (20), and the first reagent (40) and the second reagent (50) are mixed in the first body (10) or the second body (20).

Therefore, before use of the facial mask set in this embodiment, the first reagent (40) and the second reagent (50) can be separately stored in the first body (10) and the second body (20); when use of the facial mask set, a separate tool is not required, thus providing the convenience in mixing the first reagent (40) and the second reagent (50) in the facial mask set.

Further, the facial mask set in this embodiment further comprises a cut portion for forming a discharge portion (30) for discharging a third reagent (60) formed by mixing the first reagent (40) and the second reagent (50) to the outside.

More specifically, the discharge portion (30) comprises a discharge flow path (31) that forms a flow path in which the third reagent (60) moves, and a discharge opening portion (33) that opens one end of the discharge flow path (31).

Due to the discharge opening portion (33), the user can take an appropriate amount of the third reagent (60) and the discharge opening portion (33) is closed again so that the third reagent (60) is not exposed to the outside air. When the discharge opening portion (33) is opened again, the third reagent (60) can be taken, and when the third reagent (60) is applied to the face, the effect that the third reagent (60) in the same state can be used is achieved.

However, the discharge portion (30) is merely an embodiment described for convenience of explanation, and the user can make various variants and implementations in the form of the discharge portion (30) as needed, which does not limit the scope of the present invention.

For example, unlike what is shown in FIG. 3 and FIG. 4, the facial mask set in this embodiment has a cutting line formed according to the lateral distance of the upper surface of the first body (10) or the lower end of the second body (20). The user can discharge the third reagent (60) formed by mixing the first reagent (40) and the second reagent (50) contained in the first body (10) and the second body (20) by the upper surface or the lower surface of the facial mask set.

### Embodiment 4

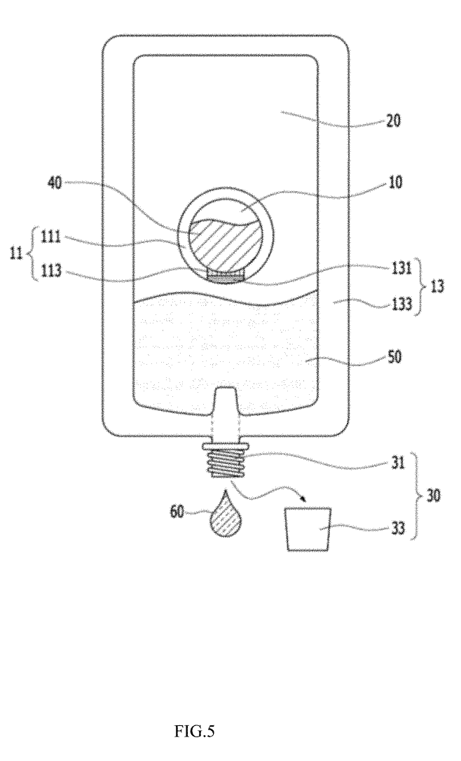

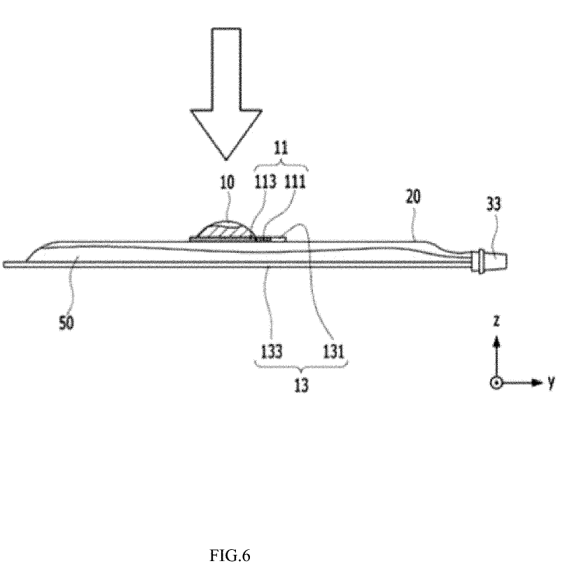

FIG. 5 shows a front view of a facial mask set according to Embodiment 4, and FIG. 6 shows a side view of the facial mask set according to Embodiment 4.

Referring to FIG. 5 and FIG. 6, the facial mask set of other embodiments comprises: a first body (10) that seals and accommodates a first reagent (40) and a second body (20) that seals and accommodates a second reagent (50).

However, unlike the facial mask sets in the embodiments shown above in FIG. 1 and FIG. 2, the first body (10) may be disposed at an upper portion of the second body (20).

The first body (10) is disposed on an outer peripheral surface of the first body (10) and comprises a first seal portion (11) for sealing the first reagent (40), and the second body (20) is disposed on an outer peripheral surface of the second body (20) and comprises a second seal portion (13) for sealing the second reagent (50).

The first seal portion (11) comprises a seal portion 1-1 (111) communicating with the second seal portion (13) and a seal portion 1-2 (113) not communicating with the second seal portion (13). The second seal portion (13) comprises a seal portion 2-1 (131) communicating with the first seal portion (11) and a seal portion 2-2 (133) not communicating with the first seal portion (11).

In this case, the sealing strength of the seal portion 1-1 (111) and the seal portion 2-1 (131) is smaller than the sealing strength of the seal portion 1-2 (113) and the seal portion 2-2 (133).

Based on this, the seal portion 1-2 (113) and the seal portion 2-2 (133) are not broken when the user presses the facial mask set from the outside, and the seal portion 1-1 (111) and the second seal portion 2-1 (131) are disposed to communicate with each other by a predetermined pressure applied externally.

More specifically, the first reagent (40) disposed in the first body (10) and the second reagent (50) disposed in the second body (20) are separated from each other before use by the user. When using the facial mask set, the user applies a pressure to the first body (10) or the second body (20) with hands, and the seal portion 1-1 (111) and the seal portion 2-1 (131) having a seal strength smaller than the above pressure are broken.

Therefore, the first reagent (40) disposed in the first body (10) at the top moves toward the second body (20) disposed below, and the first reagent (40) and the second reagent (50) are mixed in the first body (10) or the second body (20).

Therefore, before use of the facial mask set in this embodiment, the first reagent (40) and the second reagent (50) can be separately stored in the first body (10) and the second body (20); when use of the facial mask set, a separate tool is not required, thus providing the convenience in mixing the first reagent (40) and the second reagent (50) in the facial mask set.

Further, the facial mask set in this embodiment further comprises a discharge portion for discharging a third reagent (60) formed by mixing the first reagent (40) and the second reagent (50) to the outside.

More specifically, the discharge portion (30) comprises a discharge flow path (31) that forms a flow path in which the third reagent (60) moves, and a discharge opening portion (33) that opens/closes one end of the discharge flow path (31).

Due to the discharge opening portion (33), the user can take an appropriate amount of the third reagent (60) and the discharge opening portion (33) is closed again so that the third reagent (60) is not exposed to the outside air. When the discharge opening portion (33) is opened again, the third reagent (60) can be taken, and when the third reagent (60) is applied to the face, the effect that the third reagent (60) in the same state can be used is achieved.

However, the above-described discharge portion (30) is merely an embodiment described for convenience of explanation, and the user can make various modifications and implementations in the form of the discharge portion (30) as needed, which does not limit the scope of the present invention.

For example, unlike what is shown in FIG. 5 and FIG. 6, the facial mask set in this embodiment has a cutting line formed according to the lateral distance of the upper surface of the first body (10) or the lower end of the second body (20). The user can discharge the third reagent (60) formed by mixing the first reagent (40) and the second reagent (50) contained in the first body (10) and the second body (20) by the upper surface or the lower surface of the facial mask set.

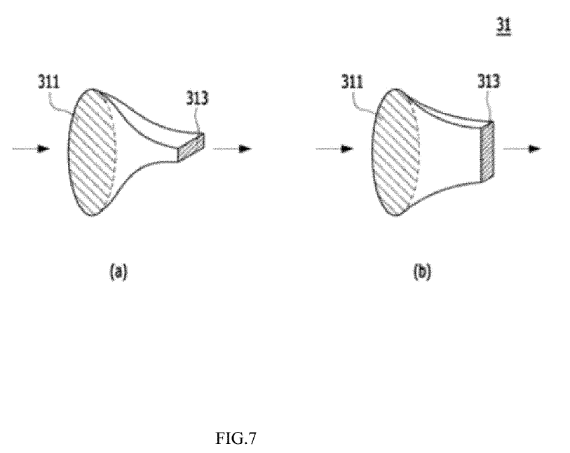

FIG. 7 shows an example of a discharge flow path of the facial mask set according to Embodiment 3 and Embodiment 4.

Referring to FIG. 7, the discharge flow path (31) in Embodiment 3 and Embodiment 4 is disposed such that the section width of an inflow portion (311) into which the third reagent flows is larger than the section width of an outflow portion (313) from which the third reagent flows out.

More specifically, the body of the discharge flow path (31) in this embodiment comprises an inflow portion (311) that provides a space in which the third reagent flows into the discharge flow path, and an outflow portion (313) that provides a space in which the third reagent flows out from the discharge flow path.

As described above, although the third reagent formed by mixing the first reagent (40) and the second reagent (50) contained in the first body (10) and the second body (20) needs to flow out of the facial mask set. The shape of the discharge flow path (31) deposited at one end of the facial mask set of Embodiment 3 and Embodiment 4 is as shown in FIG. 7(a), and the section of the outflow portion (313) may be formed to extend in the lateral direction relative to the center of gravity of the outflow portion (313). More specifically, when the shape of the section of the outflow portion (313) is based on the center of gravity thereof, the cross section of the outflow portion (313) may be set to a long rectangular shape.

On the other hand, the shape of the discharge flow path (31) is as shown in FIG. 7(b), and the section of the outflow portion (313) may be formed to extend in the vertical direction relative to the center of gravity of the outflow portion (313). More specifically, when the shape of the section of the outflow portion (313) is based on the center of gravity thereof, the vertical section of the outflow portion (313) may be set to a long rectangular shape.

Thus, since the outflow portion (313) is disposed in a shape as described above, the width of the cross-sectional area of the outflow portion (313) is smaller than the width of the cross-sectional area of the inflow portion (311), and more preferably, the width of the cross-sectional area of the outflow portion (313) is set to be about ⅓ or less of the width of the cross-sectional area of the inflow portion (311).

Since the area of the section of the outflow portion (313) of the discharge flow path (31) in this embodiment is smaller than the area of the inflow portion (311), it is possible to control amount of the third reagent (60) discharged from the first body (10) or the second body (20). Thus, the technical effect that the user can apply an appropriate amount of the third reagent (60) to the skin is achieved.

The user can make various variants and implementations in the shape of the inflow portion (311) or the outflow portion (313) of the discharge flow path (31) as needed, for example, the shape of the section can be not only rectangular but also deformed into various shapes such as a circle, a star, a triangle, a diamond, and the like, which does not limit the scope of the present invention.

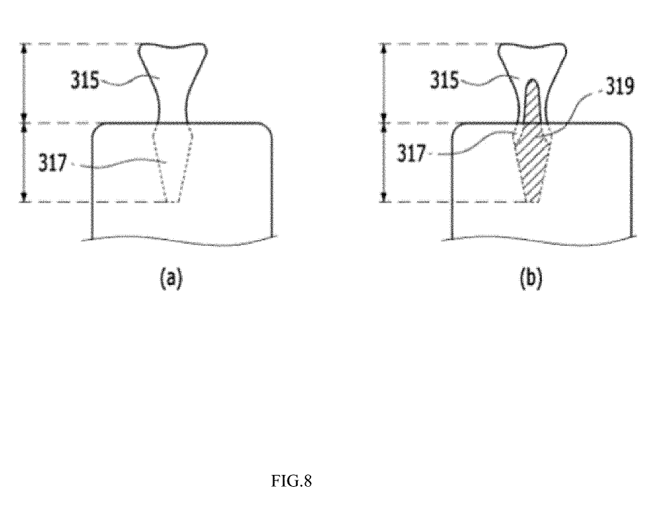

FIG. 8 shows another embodiment of the discharge flow path of the facial mask set of Embodiment 3 and Embodiment 4.

More specifically, FIG. 8 (a) shows a front view of the discharge flow path of the facial mask set of Embodiment 3 and Embodiment 4 mounted on a facial mask set, and FIG. 8(b) shows a rear view of the discharge flow path of the facial mask set of Embodiment 3 and Embodiment 4 mounted on a facial mask set.

The discharge flow path of the facial mask set shown in FIG. 8 comprises an exposed portion (317) that is provided to be exposed to the outside of the facial mask set and an insertion portion inserted in the facial mask set.

The exposed portion (315) and the insertion portion (317) may be integrally formed to function to further discharge the third reagent mixed in the facial mask to the flow path outside the facial mask set.

More specifically, the exposed portion (315) and the insertion portion (317) may each comprise a descending portion (319) that is disposed to descend from the surface to a predetermined height to move the third reagent.

When the user applies a pressure to the facial mask set, the third reagent may sequentially flow out along the descending portions (319) formed on the side of the insertion portion (317) and the exposed portion (315), and the user can directly apply to the skin the third reagent flowing out to the exposed portion (315), without using a hand.

Therefore, the effect of preventing bacterial contamination or the like caused by the third reagent contacting the user's hand can be achieved.

The material of the discharge flow path (315, 317) of this embodiment may be polypropylene (PP), polyethylene (PE), acrylonitrile-butadiene-styrene copolymer (ABS), nylon, polyethylene terephthalate-1,4-cyclohexanedimethanol ester (PETG), acrylonitrile-styrene copolymer (SAN), nitrile butadiene rubber (NBR), styrene butadiene rubber (SBR), polyurethane, elastomer or silicone resin, etc.

For the sake of hygiene, the discharge flow paths (315, 317) of the present embodiment are preferably made of an antibacterial material.

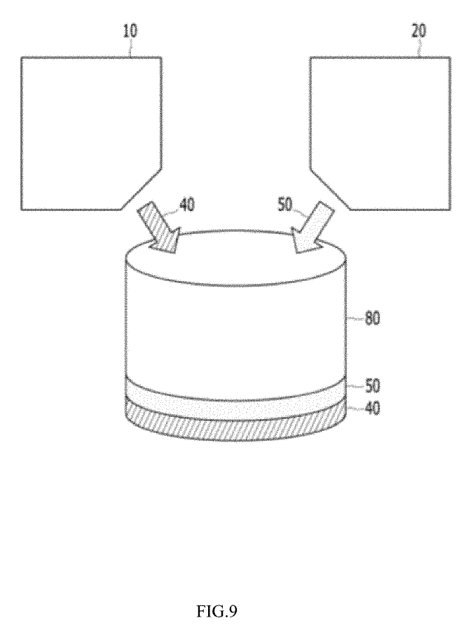

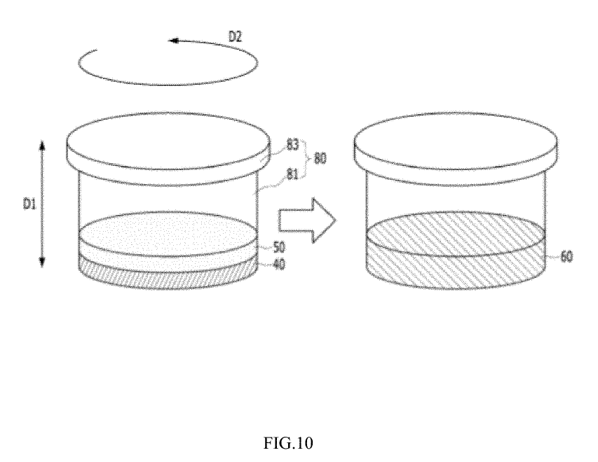

FIG. 9 shows a first body, a second body and a third body of a facial mask set according to Embodiment 5; FIG. 10 shows the facial mask set according to Embodiment 5 in which the third body contains the mixture of the first reagent and the second reagent.

In this regard, in the facial mask set of Embodiments 1-4, the first body and the second body substantially form one body, and the third reagent is formed by mixing due to the internal movement of the first reagent or the second reagent, but the facial mask set according to Embodiment may comprise: a first body (10) configured to accommodate the first reagent (40), a second body (20) configured to accommodate the second reagent (50), and a third body (80) configured to provide a disposable mixing space of the first reagent (40) discharged from the first body (10) and the second reagent (50) discharged from the second body (20).

The third body (80) of the facial mask set in this embodiment may comprise: a third body accommodating portion (81) that provides a space for accommodating and mixing the first reagent (40) and the second reagent (50); and a third body opening and closing portion (83) configured to open and close the third body accommodating portion (81).

Hereinafter, a mixing method of the facial mask in this embodiment will be described, and the first body (10) and the second body (20) may each has a cutting line, and may be torn along the perforation lines to open in part.

After the first body (10) and the second body (20) are torn apart in part, the first reagent (40) contained in the first body (10) and the second reagent (50) contained in the second body (20) may be discharged through the third body accommodating portion (81) of the third body (80).

After the first reagent (40) and the second reagent (50) in the third body accommodating portion (81) are all discharged, the third body opening and closing portion (83) configured to open and close the third body accommodating portion (81) closes one side of the third body accommodating portion (81).

The third body accommodating portion (81) in this embodiment is formed of a transparent material so that the mixing state of the first reagent (40) and the second reagent (50) contained therein being mixed into a third reagent (60) can be externally observed.

Therefore, the user can directly see the degree of mixing of the first reagent (40) and the second reagent (50) accommodated in the third body accommodating portion (81).

Further, an indicator having the same color with the third reagent (60) formed after mixing the first reagent (40) and the second reagent (50) in the third body accommodating portion (81) is also provided (not shown).

Therefore, the user can more clearly determine the degree of mixing of the first reagent (40) and the second reagent (50); when the color of the mixture of the first reagent (40) and the second reagent (50), accommodated in the third body accommodating portion (81), becomes the same as the color of the third reagent (60) described above, the mixture can be used and the third reagent (60) can be applied to the face at a more proper time.

Thereafter, the user makes the third body (80) cause a relative displacement change of the first reagent (40) and the second reagent (50) in various directions, so that the first reagent (40) and the second reagent (50) accommodated in the third body (80) are mixed.

For example, as the direction in which the user agitates the third body (80), the third body (80) may be agitated in a first direction (D1), or in a second direction (D2), or in both the first direction (D1) and the second direction (D2).

The first direction (D1) may be a vertical direction, and the second direction (D2) may be a clockwise direction or a counterclockwise direction with respect to the center of the upper surface of the third body (80).

By agitating the third body accommodating portion (81), the user can mix the first reagent (40) and the second reagent (50) accommodated inside the third body accommodating portion (81) more efficiently than in the conventional manner.

On the other hand, as in the above embodiment, the first reagent (40) and the second reagent (50) are accommodated in the first body (10) and the second body (20) which are separated from each other, the first body (10) and the second body (20) can discharge the regents to an additional new third body (80) to cause a mixing action, but it is also possible that the first reagent (40) has been contained in the third body (80), and then the second reagent (50) is additionally discharged into the third body (80), so that the first reagent (40) and the second reagent (50) are mixed. In this case, the accommodating positions of the first reagent (40) and the second reagent (50) may be exchanged.

Further, in addition to the method of agitating the third body (80), the user may use a displacement change generating means (not shown). At least a portion of the displacement change generating means is inserted into the third body (80) to cause the relative displacement change of the first reagent (40) and the second reagent (50) so that the first reagent (40) and the second reagent (50) are mixed.

For example, the displacement change generating means (not shown) is provided with one end inserted into the third body (80) and the other end held by the user. the user holds the other end of the displacement change generating means (not shown) to generate a power, and the generated power is transmitted to the end, inserted into the third body (80), of the displacement change generating device, thereby generating displacement change of the first reagent (40) and the second reagent (50).

Therefore, the first reagent (40) and the second reagent (50) can be more easily mixed.

However, for convenience of explanation, a method of causing displacement change of the first reagent (40) and the second reagent (50) is described, and the user can select various variants and implementations of the method of cause displacement change of the first reagent (40) and the second reagent (50) as needed, which are not intended to limit the scope of the invention.

On the other hand, the first reagent (40) and the second reagent (50) of this embodiment can be provided as follows.

More specifically, the ratio of the first reagent (40) to the second reagent (50) is 95:5 to 50:50, preferably 80:20 to 50:50, more preferably 70:30 to 60:40 by volume.

The first reagent (40) is purified water or a cosmetic liquid, and is emulsified when mixed with the second reagent (50), and gels after a certain period of time.

Purified water can be used as a solvent.

The amount of the above solvent used in the first reagent (40) according to the present invention is not particularly limited as long as the effect of the present invention can be achieved, and the use amount of the solvent is as high as about 100 wt %, preferably 70 wt %, more preferably 87-97 wt %, with respect to the total weight of the first reagent (40).

A metal ion chelating agent is not particularly limited as long as the effect of the present invention can be achieved, and any metal ion chelating agent known in the art may be used.

The metal ion chelating agent is a component which combines with a metal ion which adversely affects the stability or properties of the cosmetic and forms a composite salt to inactivate the metal ion, and functions to prevent the metal ion from being precipitated by another reagent.

Typically, the metal ion chelating agent may be one component selected from a group consisting of condensed phosphate, ethylenediaminetetraacetic acid (EDTA), disodium EDTA, nitrilotriacetic acid, dihydroxyethylglycine, gluconic acid, citric acid, and tartaric acid, and preferably uses disodium EDTA.

The amount of the above metal ion chelating agent used in the first reagent (40) according to the present invention is not particularly limited as long as the effect of the present invention can be achieved, and the use amount of the metal ion chelating agent is preferably 0.01-1 wt %, more preferably 0.01-0.06 wt %, most preferably 0.01 wt %, with respect to the total weight of the first reagent (40).

A wrinkle removal agent, a whitening agent, a humectant and the like as effective active ingredients can also be used.

A wrinkle removal functional material may be one selected from a group comprising adenosine, retinol, retinyl palmitate and polyethoxylated retinamide, and preferably uses adenosine.

The amount of the above wrinkle removal functional material used in the first reagent (40) according to the present invention is not particularly limited as long as the effect of the present invention can be achieved, and the use amount of the wrinkle removal functional material is preferably 0.02-0.06 wt %, more preferably 0.04 wt %, with respect to the total weight of the first reagent (40).

* A whitening functional raw material is one selected from a group comprising niacinamide, broussonetia extract, ascorbyl glucoside, ascorbyl tetraisopalmitate, arbutin, α-Bisabolol, ethyl ascorbyl ether, and oil soluble licorice (glycyrrhiza) extract, and preferably uses niacinamide.

The amount of the above whitening functional material used in the first reagent (40) according to the present invention is not particularly limited as long as the effect of the present invention can be achieved, and the use amount of the whitening functional material is preferably 1.8-2.2 wt %, more preferably 2.0 wt %, with respect to the total weight of the first reagent (40).

A humectant is not particularly limited as long as the effect of the present invention can be achieved, and any humectant known in the art can be used.

Generally, the humectant is one selected from a group consisting of glycerin, propylene glycol, dipropylene glycol, butanediol, isopropyl glycol, pentanediol, arginine, and mixtures thereof;

The amount of the above metal ion chelating agent used in the first reagent (40) according to the present invention is not particularly limited as long as the effect of the present invention can be achieved, and the use amount of the metal ion chelating agent is preferably 0.1-25 wt %, more preferably 2.0-6.0 wt %, most preferably 4.0 wt %, with respect to the total weight of the first reagent (40).

A surfactant is not particularly limited as long as the effect of the present invention can be achieved, and any surfactant known in the art can be used.

Typically, the surfactant may be one selected from a group consisting of polyglycerin fatty acid esters, polyethylene glycol fatty acid esters, polyoxyethylene hydrogenated castor oil, polyoxyethylene alkyl ethers, polyoxyethylene glycerin fatty acid esters, polyoxyethylene polyoxypropylene alkyl ether and mixtures thereof, and preferably selected from of a group consisting of PEG-60 hydrogenated castor oil, hydrogenated polyisobutylene, hydrogenated polydecene, ethylhexyl palmitate, squalene, macadamia nut oil, olive oil, dwarf seed oil, refined grape seed oil, caprylic/capric triglyceride, cyclomethicone, polydimethylsiloxane, PEG-100 stearate, glyceryl stearate, stearyl alcohol and mixtures thereof.

The surfactant contained in the first reagent (40) according to the present invention is more preferably PEG-60 hydrogenated castor oil.

The amount of the above surfactant used in the first reagent (40) according to the present invention is not particularly limited as long as the effect of the present invention can be achieved, and the use amount of the surfactant is preferably 0.001-5 wt %, more preferably 0.2-0.4 wt %, most preferably 0.3 wt %, with respect to the total weight of the first reagent (40).

The surfactant contained in the second reagent (50) according to the present invention is used for emulsifying an emollient when mixed with the first reagent (40), and is not limited to the above examples, and all surfactants used in the cosmetic industry can be used.

The surfactant contained in the second reagent according to the present invention is more preferably PEG-8 glyceryl isostearate.

The amount of the above surfactant used in the second reagent (50) according to the present invention is not particularly limited as long as the effect of the present invention can be achieved, and the use amount of the surfactant is preferably 1.0-25 wt % with respect to the total weight of the second reagent (50). When the use amount of the surfactant is less than 1 wt %, the emulsifying effect is poor, and the separation of the first reagent (40) and the second reagent (50) occurs, and when the use amount of the surfactant is more than 25 wt %, the moisturizing effect is lowered.

The preservative is not particularly limited as long as the effect of the present invention can be achieved, and any one of the preservatives known in the art can be used. The preservative may be typically at least one selected from a group consisting of ethylhexyl glycerin, phenoxyethanol, methylparaben, and Sensciva SC 50, or may be a combination of two of the components listed above. Preferably, ethylhexyl glycerin and phenoxyethanol are used in combination.

The amounts of the above preservative used in the first reagent (40) and the second reagent (50) according to the present invention are not particularly limited as long as the effect of the present invention can be achieved, and the use amount of the preservative is preferably 0.02-2 wt %, more preferably 0.2-0.06 wt %, most preferably 0.4 wt %, respectively with respect to the total weight of the first reagent (40) and the total weight of the second reagent (50). In the case of two or more preservatives are used in combination, the combination is a combination of preservative 1 (0.01-1 wt %) and preservative 2 (0.01-1 wt %), preferably a combination of preservative 1 (0.05-0.15 wt %) and preservative 2 (0.1-0.5 wt %), and more preferably a combination of preservative 1 (0.1 wt %) and preservative 2 (0.3 wt %). Most preferably, 0.1 wt % of ethylhexyl glycerin and 0.3 wt % of phenoxyethanol are used in combination.

An emollient is not particularly limited as long as the effect of the present invention can be achieved, and any oil commonly used in the cosmetic industry can be used.

Typically, the emollient may be at least one selected from a group consisting of silicone oils, mineral oils, animal oils, vegetable oils, and ester oils, more preferably a vegetable oil, and most preferably olive oil.

The amount of the above emollient used in the second reagent (50) according to the present invention is not particularly limited as long as the effect of the present invention can be achieved, and the use amount of the emollient is preferably 65-95 wt %, with respect to the total weight of the second reagent (50).

The moisturizing effect is lowered when the use amount of the emollient is less than 65 wt %, and the gel is not formed well when the use amount of the emollient exceeds 95 wt %.

A thickener contained in the second reagent (50) according to the present invention is used for thickening the second reagent (50) containing an oily emollient to prevent powder components, such as a gel forming agent, a gel retarder and a gelling agent, contained in the second reagent (50) from precipitating or floating, and is not particularly limited as long as the effect of the present invention can be achieved, and any thickener known in the art can be used.

Typically, the thickener is at least one selected from a group consisting waxes, polymers, and silicas, and preferably selected from a group consisting of natural polymers (such as acacia gum, carrageenan, galactan, agar, guar gum, pectin, mannan, wheat starch, rice starch, corn starch, potato starch, xanthan gum, albumin, collagen and gelatin); semi-synthetic polymers (such as ethylcellulose, carboxymethylcellulose or salts thereof, carboxymethylethylcellulose or salts thereof, crystalline cellulose, hydroxyethylcellulose, hydroxymethylcellulose, methylhydroxypropylcellulose; and synthetic polymers (such as carboxyvinyl polymer, sodium polyacrylate, polyvinyl alcohol and acrylate/alkyl acrylate cross-linked polymer); the thickener most preferably is silica dimethyl silane ester.

The amount of the above thickener used in the second reagent (50) according to the present invention is not particularly limited as long as the effect of the present invention can be achieved, and the use amount of the thickener is preferably 0.5-20 wt % with the total weight of the second reagent (50). When the use amount of the thickener is less than 0.5 wt %, the above-mentioned powder components may precipitate, and the first reagent (40) and the second reagent (50) cannot form a gel or form a gel with low strength when mixed. When the use amount of the thickener exceeds 20 wt %, the second reagent (50) is highly viscous, and is difficulty mixed with the first reagent (40).

A gel forming agent contained in the second reagent (50) according to the present invention is contained in the second reagent (50). Since the gel forming agent functions as a thickener to be hydrated with purified water or a cosmetic liquid of the first reagent (40), the gel forming agent reacts with a gelling agent to increase viscosity and form a gel when the first reagent (40) and the second reagent (50) are mixed. The gel forming agent is not particularly limited as long as the effect of the present invention can be achieved, and any gel forming agent known in the art can be used.

The above gel forming agent is preferably sodium alginate or potassium alginate, more preferably sodium alginate.

The amount of the above gel forming agent used in the second reagent (50) according to the present invention is not particularly limited as long as the effect of the present invention can be achieved, and the use amount of the gel forming agent is preferably 1-25 wt %, with respect to the total weight of the second reagent (50).

When the use amount of the gel forming agent is less than 1 wt %, the viscosity is low, and the dripping is severe when the mask is applied to the skin, a gel cannot be formed, and the strength of the gel is low. When the use amount of the gel forming agent exceeds 25 wt %, the viscosity is too high, the first reagent (40) and the second reagents (50) are difficulty mixed or the surface is uneven when the mixture is applied to the skin, and a gel is hardly formed due to aggregation.

A gel retarder contained in the second reagent (50) according to the present invention is used for adjusting the curing reaction speed of the gel when the first reagent (40) and the second reagent (50) are mixed and applied to the skin. The gel retarder is not particularly limited as long as the effect of the present invention can be achieved, and any gel retarder known in the art can be used.

Typically, the gel retarder may be one selected from a group comprising tetrapotassium pyrophosphate, and preferably uses tetrapotassium pyrophosphate.

### EXAMPLES

Specific examples are presented below to facilitate the understanding of the present invention. However, the following examples are provided only to more easily understand the present invention, and the contents of the present invention are not limited by these examples.

The liquid-phase first reagent and the liquid-phase second reagent are prepared based on the components shown in Tables 1 and 2 below.

TABLE 1
First reagent

| No. | Material name | Type | Content (wt %) |
|---|---|---|---|
| 10 | Purified water | Solvent | 93.25 |
| 20 | EDTA | Metal ion chelating agent | 0.01 |
| 30 | Adenosine | Wrinkle removal | 0.04 |
| | | functional material | |
| 40 | Niacinamide | Whitening functional | 2.00 |
| | | material | |
| 50 | Dipropylene glycol | Solvent, humectant | 4 |
| 60 | PEG-60 hydrogenated | Surfactant, solubilizer, | 0.3 |
| | castor oil | emulsifier | |
| 70 | Ethylhexylglycerin | Preservative | 0.1 |
| 80 | Phenoxyethanol | Preservative | 0.3 |

The specific preparation process is as follows.
1. Add the raw material 10 to a main tank, and then add the raw materials 20-80, and stir.
2. Degas in vacuum, filter and store.

TABLE 2
Second reagent

| | | | Content |
|---|---|---|---|
| No. | Material name | Type | (wt %) |
| 10 | Olive oil | Emollient | 65 |
| 20 | PEG-8 glyceryl isostearate | Surfactant, emulsifier, | 10 |
| | | solubilizer | |
| 30 | Ethylhexylglycerin | Preservative | 0.1 |
| 40 | Phenoxyethanol | Preservative | 0.3 |
| 50 | Silica dimethyl silane ester | Thicker, dispersant | 7 |
| 60 | Sodium alginate | Gel forming agent, thicker, | 10 |
| | | dispersant | |
| 70 | Tetrapotassium | Gel retarder | 3.8 |
| | pyrophosphate | | |
| 80 | Calcium sulfate | Gelling agent | 3.8 |

The specific preparation process is as follows.
1. Add the raw material 10 to a main tank, and then add the raw materials 20-50, and stir.
2. Slowly add raw materials 60-80 in the manufacturing process 1 and stir.
3. Degas in vacuum, filter and store.

### Experimental Example

In the experimental example, the first reagent and the second reagent shown in Embodiment 1 are immediately mixed and used before use. A bag type container in which the first reagent and the second reagent are mixed is shaken up and down by a hand, or the first reagent and the second reagent are stirred in a spherical container to be mixed simply, and then a gel is formed within 30 seconds after mixing. In order to evaluate the user experience of the facial mask cosmetic composition of the present invention, the user experience of 20 women over the age of 30 after applying the facial mask composition is evaluated.

The facial mask cosmetic composition of the present invention can be applied to the skin after mixing the first reagent and the second reagent at ratios 95:5, 90:10, 85:15, 80:20, 75:25, 70:30, 65:35, 60:40, 55:45 and 50:50, respectively. After being applied to the skin for 20 minutes, the facial mask cosmetic composition is peeled off from the skin, and items such as fluidity, gel strength, and moisturizing effect are evaluated.

TABLE 3
<Evaluation criteria>

| Mixing ratio (first | | | |
|---|---|---|---|
| reagent:second | Evaluation of use | | |
| reagent) | Fluidity | Gel strength | Moisturizing effect |
| 95:5 | X | X | ◯ |
| 90:10 | X | X | ◯ |
| 85:15 | X | Δ | ◯ |
| 80:20 | Δ | Δ | ◯ |
| 75:25 | Δ | ◯ | ◯ |
| 70:30 | ◯ | ◯ | ◯ |
| 65:45 | ◯ | ◯ | ◯ |
| 60:40 | ◯ | ◯ | ◯ |
| 55:45 | ◯ | Δ | Δ |
| 50:50 | ◯ | Δ | Δ |

◯: Excellent
Δ: Normal
X: Poor

As shown in the Table 3 above, when the use amount of the first reagent is 1.5 times or more as much as the use amount of the second reagent, the moisturizing effect is excellent. When the first reagent and the second reagent are combined at a ratio of 70:30 or less, the dripping hardly occurs. When the first reagent and the second reagent are combined at a ratio of 75:25 or less, the gel strength is excellent. As the amount of the first reagent containing the humectant or the like increases, the moisturizing effect is improved, and as the amount of the second reagent containing the emollient, the surfactant, and the like increases, the gel strength becomes strong and the fluidity is lowered.

The description of the present invention is intended to be illustrative of the preferred embodiments of the invention and provide embodiments to illustrate the present invention and enable one of ordinary skill in the art to make and use the present invention. The description completed in this way is not intended to limit the present invention to the specific terms presented.

Therefore, the present invention has been described in detail with reference to the embodiments of the present invention, and substitutes, modifications and changes of these embodiments will be apparent to those skilled in the art without departing from the scope of the invention. In other words, in order to achieve the expected effect of the present invention, all the functional blocks shown in the figures are individually included, or all the steps shown in the drawings are not necessarily performed in the order shown. It should be noted that the present invention may fall within the technical scope of the invention described in the claims, and is not limited to the above embodiments.

### INDUSTRIAL APPLICABILITY

The present invention relates to a facial mask which is industrially applicable to the field of cosmetics.

I claim:

1. A peel-off facial mask cosmetic composition, comprising: a liquid-phase first reagent containing purified water; and a liquid-phase second reagent containing an oily emollient, a surfactant, a gel forming agent, a gelling agent and a gel retarder,
wherein the gel forming agent, the gelling agent and the gel retarder contained in the second reagent are not gelled by the oily emollient and are present in an inactive state.

2. The peel-off facial mask cosmetic composition according to claim 1, wherein the ratio of the first reagent to the second reagent is 95:5 to 50:50 by volume.

3. The peel-off facial mask cosmetic composition according to claim 1, wherein the content of the purified water is 70 wt % or more based on the total weight of the first reagent, the content of the oily emollient is 65-95 wt % based on the total weight of the second reagent, and the content of the surfactant is 1-25 wt % based on the total weight of the second reagent.

4. The peel-off facial mask cosmetic composition according to claim 1, wherein the oily emollient is an oil.

5. The peel-off facial mask cosmetic composition according to claim 1, wherein the first reagent further comprises at least one selected from a group consisting of a metal ion chelating agent, a wrinkle removal functional raw material, a whitening functional raw material, a humectant, a surfactant, and a preservative.

6. The peel-off facial mask cosmetic composition according to claim 5, wherein based on the total weight of the first reagent, the content of the metal ion chelating agent is 0.001-1 wt %, the content of the wrinkle removal functional raw material is 0.02-0.06 wt %, the content of the whitening functional raw material is 1.8-2.2 wt %, the content of the humectant is 0.1-25 wt %, and the content of the surfactant is 0.001-5 wt %.

7. The peel-off facial mask cosmetic composition according to claim 5, a peel-off facial mask cosmetic composition, wherein the metal ion chelating agent is one selected from a group consisting of condensed phosphate, ethylenediaminetetraacetic acid (EDTA), disodium EDTA, nitrilotriacetic acid, dihydroxyethylglycine, gluconic acid, citric acid, and tartaric acid;
the wrinkle removal functional material is one selected from a group consisting of adenosine, retinol, retinyl palmitate and polyethoxylated retinamide;
the whitening functional raw material is one selected from a group consisting of niacinamide, broussonetia extract, ascorbyl glucoside, ascorbyl tetraisopalmitate, arbutin, α-Bisabolol, ethyl ascorbyl ether, and oil soluble licorice (glycyrrhiza) extract;
the humectant is one selected from a group consisting of glycerin, propylene glycol, dipropylene glycol, butanediol, isopropyl glycol, pentanediol, arginine, and mixtures thereof;
the surfactant is one selected from a group consisting of polyglycerol fatty acid esters, polyethylene glycol fatty acid esters, polyoxyethylene hydrogenated castor oil, polyoxyethylene alkyl ethers, polyoxyethylene glycerin fatty acid esters, polyoxyethylene polyoxypropylene alkyl ethers, and mixtures thereof.

8. The peel-off facial mask cosmetic composition according to claim 7, wherein the metal ion chelating agent is disodium EDTA;
the wrinkle removal functional material is adenosine;
the whitening functional material is niacinamide;
the humectant is dipropylene glycol;
the surfactant is PEG-60 hydrogenated castor oil.

9. The peel-off facial mask cosmetic composition according to claim 1, wherein the second reagent further comprises at least one component selected from a group consisting of a preservative and a thickener.

10. The peel-off facial mask cosmetic composition according to claim 9, wherein based on the total weight of the second reagent, the content of the thickener is 0.5-20 wt %, the content of the gel forming agent is 1-25 wt %, the content of the gel retarder is 0.01-10 wt %, and the content of the gelling agent is 0.01-10 wt %.

11. The peel-off facial mask cosmetic composition according to claim 9, wherein the surfactant is one selected from a group consisting of polyglycerol fatty acid ester fatty acid esters, polyoxyethylene hydrogenated castor oil, polyoxyethylene alkyl ethers, polyoxyethylene glycerin fatty acid esters, polyoxyethylene polyoxypropylene alkyl ethers, and mixtures thereof;
the thickener is at least one selected from a group consisting of waxes, polymers, and silicas.

12. The peel-off facial mask cosmetic composition according to claim 11, wherein the surfactant is PEG-8 glyceryl isostearate;
the thicker is silica dimethyl silane ester.

13. A facial mask set, comprising:
a first body providing a space for accommodating a first reagent;
a second body extending over or below the first body and providing a space for accommodating a second reagent; and
a discharge portion for discharging a third reagent formed after the first reagent and the second reagent are mixed;
the first reagent is provided in a liquid phase containing purified water;
the second reagent is provided in a liquid phase containing an oily emollient, a gel forming agent, a gelling agent, and a gel retarder;
wherein the gel forming agent, the gelling agent and the gel retarder contained in the second reagent are not gelled by the oily emollient and are present in an inactive state.

14. The facial mask set according to 13, wherein the first body is disposed on an outer peripheral surface of the first body and comprises a first seal portion for sealing the first reagent;
the second body is disposed on an outer peripheral surface of the second body and comprises a second seal portion for sealing the second reagent.

15. The facial mask set according to claim 14, wherein the first seal portion comprises a seal portion 1-1 that communicates with the second seal portion and a seal portion 1-2 that does not communicate with the second seal portion;
the second seal portion comprises a seal portion 2-1 that communicates with the first seal portion and a seal portion 2-2 that does not communicate with the first seal portion.

16. The facial mask set according to claim 15, wherein the sealing strength of the seal portion 1-1 and the seal portion 2-1 is smaller than the sealing strength of the seal portion 1-2 and the seal portion 2-2.

17. The facial mask set according to claim 13, wherein the discharge portion comprises: a discharge flow path for providing a flow path in which the third reagent flows, and a discharge opening portion which opens one end of the discharge flow path;
the cross-sectional area of an inflow portion of the discharge flow path into which the third reagent flows is larger than the cross-sectional area of an outflow portion from which the third reagent flows out.

18. The facial mask set according to claim according to claim 13, wherein the discharge portion is formed by cutting a first cut portion provided on a right side or a left side surface of the facial mask set and a second cut portion provided on an upper surface or a lower surface of the facial mask set.

19. The facial mask set according to claim according to claim 13, wherein a lower surface of the second body is formed into a downwardly protruding shape;
the discharge portion is formed by cutting a first cut portion provided on a left side of the facial mask set and a second cut portion provided on a right side of the facial mask set, and the discharge portion penetrates through the lower part of the second body.

20. A facial mask set, comprising:
a first body for accommodating a first reagent;
a second body for accommodating a second reagent; and
a discharge portion for providing a space for a third reagent formed after the first reagent and the second reagent are mixed;
wherein after the first reagent and the second reagent are injected into the third body, the third reagent is formed by a relative displacement change of the first reagent and the second reagent, and the third reagent is a cream-type emulsion with viscosity higher than the viscosity of the first reagent and the second reagent and is gelled over time;
the first reagent is provided in a liquid phase containing purified water;
the second reagent is provided in a liquid phase containing an oily emollient, a gel forming agent, a gelling agent, and a gel retarder;
wherein the gel forming agent, the gelling agent and the gel retarder contained in the second reagent are not gelled by the oily emollient and are present in an inactive state.

21. A facial mask set, comprising:
a first body for accommodating a first reagent; and
a second body for accommodating a second reagent and providing a space for a third reagent formed after the first reagent and the second reagent are mixed;
wherein after the first reagent is injected into the second body, the third reagent is formed by a relative displacement change of the first reagent and the second reagent, and the third reagent is a cream-type emulsion with viscosity higher than the viscosity of the first reagent and the second reagent and is gelled over time;
the first reagent is provided in a liquid phase containing purified water;
the second reagent is provided in a liquid phase containing an oily emollient, a gel forming agent, a gelling agent, and a gel retarder;
wherein the gel forming agent, the gelling agent and the gel retarder contained in the second reagent are not gelled by the oily emollient and are present in an inactive state.

22. The facial mask set according to claim 20, wherein the relative displacement change of the first reagent and the second reagent is generated by agitation of the third body.

23. The facial mask set according to claim 20, wherein the third body comprises:
a third body accommodating portion for accommodating the first reagent and the second reagent; and
a third body opening and closing portion for opening or closing at one side of the accommodating portion.
